(19) United States
(12) Patent Application Publication
(10) Pub. No.: US 2024/0415692 A1
(43) Pub. Date: Dec. 19, 2024

(54) LOGOPAEDIC TREATMENT DEVICE

(71) Applicant: CODONIS AG, Muttenz (CH)

(72) Inventors: Stefan STÜBINGER, Binningen (CH); Fabio CIRILLO, Ormalingen (CH)

(21) Appl. No.: 18/704,969

(22) PCT Filed: Oct. 27, 2022
(86) PCT No.: PCT/EP2022/080133

§ 371 Date: Apr. 26, 2024

(30) Foreign Application Priority Data
Oct. 28, 2021 (DE) 10 2021 128 188.1

(51) Int. Cl. A61F 5/58

(52) U.S. Cl. CPC A61F 5/58

(57) ABSTRACT

A logopaedic treatment device has a U-shaped basic shape to be inserted into the mouth. The logopaedic treatment device includes a plate segment for partially covering a U-shaped basic shape to be inserted into the mouth, a plate segment for partially covering the rows of teeth in the upper and lower jaw, and, on the side facing away from the teeth, at least one projection, in particular an arrangement of a number of projections, for lip stimulation. The bite strip defines a bite-on plane and the projections are arranged both above and below the bite-on plane.

### FIELD OF THE INVENTION

Exemplary embodiments of the present invention relate to a logopaedic treatment device, also known and hereinafter referred to as LCT trainer (lips, cheeks and tongue).

### BACKGROUND OF THE INVENTION

The development of the LCT trainer is the result of many years of research by Dr. h.c. Codoni. The LCT trainer is used as an aid in speech therapy, dentistry, orthodontics, pediatrics, ENT, phoniatrics, and oral and maxillofacial surgery.

The LCT trainer is primarily used to train the tongue position and lip closure. The trainer can also be used in a familiar way for orofacial disorders, to support healthy nasal breathing, to break various habits, to stabilize the function of the mimic muscles, to reduce hypersalivation and to improve the pronunciation of primary sounds such as S, SH and Z and secondary sounds such as L, N, D and T.

The well-known LCT trainer is made of elastic silicone in the shape of a bite splint. A basic shape can be shaped by customization such as cutting or grinding.

An alternative form of an LCT trainer as an extension of the LCT trainer by Dr. h.c. Codoni is disclosed in DE 20 2017 000 128 U1.

US 2020/0 229 750 A1 is also known from the prior art. This is a training device that is purely oriented to the upper jaw and partially fixed, which only enables stimulation of the adjacent anatomical parts of the upper jaw. When opening the mouth, there is a risk of unintentional release of the training device, which is perceived as disturbing and distracting. In addition, the device can easily be manipulated unintentionally with the tongue.

US 2020/0 215 384 A1 has a massive projection in the lip closure area on the bite-on plane, which impairs functional lip closure. In addition, smaller projections with reference sign 38 are arranged below the bite-on plane for the sole and explicit stimulation of the musculus mentalis. Due to the position of the projections, the stimulation is therefore limited to this muscle area in the front region of the lower jaw. Overall stimulation of the mouth ring muscle and/or the muscles of both jaws, including the cheek muscles, is definitely not achieved by the subject matter of this publication.

Exemplary embodiments of the present invention to improve myofunctional stimulation in the orofacial area, in particular the lip and tongue function of the LCT trainer and, in particular, to train lip closure. In combination with an integrated or external measurement and evaluation of sensor data, it should be possible to derive personalized therapies and their success. It is also possible to deliver active ingredients through the LCT.

### SUMMARY OF THE INVENTION

A logopaedic treatment device according to the invention has the U-shaped base shape of a bite splint. It is used for insertion into the mouth. The logopaedic treatment device has a plate segment for partially covering a row of teeth, in particular the rows of teeth of the upper and lower jaw. The logopaedic treatment device also has at least one bite strip for the teeth of the upper and lower jaw to bite on.

Preferably, the bite strip in combination with the plate segment offers the possibility of modular adaptation to upper and lower jaw situations of different sizes.

According to the invention, the plate segment has at least one projection on the side facing away from the tooth, i.e., in the direction of the lips. In particular, this may be an arrangement of several projections. The projection or the arrangement of several projections serve to stimulate the lips or cheek/face muscles.

By stimulating the lips or cheek/face muscles, an existing logopaedic treatment device-a so-called LCT trainer—can be supplemented with additional functions.

The plate segment can advantageously have at least 5, preferably at least 10 projections for lip stimulation.

The projections can preferably have a height of at least 0.5 mm, preferably greater than 2.0 mm.

The projections can also preferably be cylindrical, dome-shaped or frustoconical.

To further improve lip stimulation, the projections can have movable, in particular rotatable or linearly movable, elements.

The projections can have sensor elements to monitor the progressive success of the training and/or the state of health in the mouth. The treatment device can also have actuators to support the training.

Furthermore, the logopaedic treatment device can have a retaining surface or recess for clamping or latching holding of an active substance depot or a flavoring substance depot, in particular a plate-shaped active substance or flavoring substance depot. The logopaedic treatment device can therefore be used both as an applicator and for stimulation. This improves the transport of active ingredients by stimulating blood circulation and saliva flow and/or additionally stimulates the sense of taste.

The logopaedic treatment device, in particular the projections, may alternatively or additionally comprise an active substance and/or a flavoring agent and/or an indicator substance and/or a biomarker.

The logopaedic treatment device can also advantageously have a telescopic extension for adapting the logopaedic treatment device to the size of a set of teeth. The extension can, for example, relate to the bite strip and/or the plate segment.

The logopaedic treatment device can be designed as a single piece, in particular monolithically.

Alternatively, the logopaedic treatment device can have a modular structure. It is particularly preferable for the projection(s) to be interchangeably arranged as interchangeable modules on the plate segment. Alternatively, other elements, such as the bite strip or other projections, can also be designed to be interchangeable or extendable.

The logopaedic treatment device can be made of a material with a Shore hardness A 40-80, preferably below 70, at least in some areas.

Advantageously, the plate segment can have areas with different surface roughness, especially beyond the projections and particularly preferably on the side of the plate segment facing the teeth.

### BRIEF DESCRIPTION OF THE DRAWINGS

In the following, the invention is described in more detail with reference to the drawing by means of exemplary embodiments, wherein further advantageous variants and embodiments are also discussed. It should be emphasized that the exemplary embodiments discussed below are not intended to describe the invention exhaustively, but that variants and equivalents not shown are also feasible and fall within the scope of the claims, wherein:

FIG. 1 shows a perspective view of an LCT trainer according to the invention;

FIG. 2 shows a perspective rear view of the LCT trainer;

FIG. 3 shows a front view of the LCT trainer;

FIG. 4 shows a top view of the LCT trainer; and

FIG. 5 shows a side view of the LCT trainer

### DETAILED DESCRIPTION

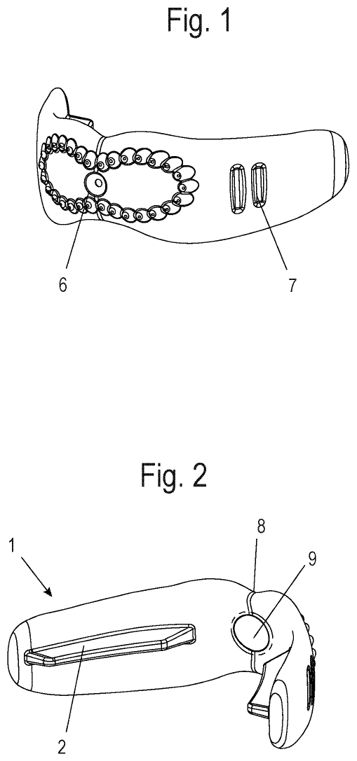

FIG. 1 shows a logopaedic treatment device. Such a device is also known in specialist circles as LCT trainer 1 (lips, cheeks and tongue trainer). The basic treatment device was developed by Ms. Dr. h.c. Codoni and has been successfully on the market for several years.

The LCT trainer 1 has a U-shaped basic shape having a plate segment 3 and two bite strips 2 projecting into the U-shape. The bite strips 2 are used to support teeth on both sides. In the context of the present invention, the side of the plate segment 3 from which the bite strips project is described as the “tooth side” and the side of the plate segment opposite this side is described as the “lip side”.

The LCT trainer 1 consists of a flexible plastic material, in particular a thermoplastic material and/or a thermoplastic elastomer.

The plate segment can have a demolding-related burr of less than 0.3 mm, preferably less than 0.2 mm, all the way around. Alternatively, this burr can also be removed in a reworking step and have a corresponding material abrasion point.

Based on this basic form, the further development of the LCT trainer consists of several elements to improve lip stimulation.

The LCT trainer has one or more projections 5 for lip stimulation, which project from the plate segment 3. These projections 5 are preferably arranged in at least one straight or curved row.

The projections 5 can each be drop-shaped, ovaloid, and/or cylindrical and each have a central recess. The projections 5 can be arranged in rows in an annular basic shape 10.

The arrangement 10 of a plurality of projections 5 can also hold a non-displayed sensor arrangement and/or a non-displayed active ingredient system, for example in a plate-shaped design, in a clamping, clinging, hooking, and/or latching manner on a retaining surface 4 of the LCT trainer 1. For this purpose, the plate shape should preferably have a lower plate thickness than the height of at least some of the projections 5. In addition, it is recommended that the plate shape be bendable for better, preferably flat, support on the retaining surface 4.

The retaining surface 4 can alternatively or additionally serve to hold a sensor arrangement and/or an active ingredient system for the arrangement of a label.

Furthermore, the plate segment 3 has a point-shaped central surface 6 on the lip side in a central arrangement on the plate segment 3. This can, for example, be formed by an injection point during manufacture and divides the plate segment 3 into two wings. The central surface 6 is used to check the alignment and fit of the LCT trainer in the mouth.

On the tooth side, the plate segment 3 also has a central surface 9, preferably in a point or oval shape. This central surface has a spherical or conical shape and can have a different surface quality, preferably a different surface roughness, to the neighboring surfaces, in particular the wings of the plate segment.

This serves to fix a device for tongue stimulation in order to create greater acceptance of the LCT trainer 1 among users, especially children and adolescents.

The respective wings of the plate segment 3 have further projections 7. Alternatively, notches or recesses can also be provided. These serve to improve the grippability of the LCT trainer 1 and are preferably at least 15%, particularly preferably more than 30%, in relation to the total length of the respective wing, away from the center of the LCT trainer 1 in which the two wings meet.

Starting from the two-dimensional view of the U-shape of the LCT trainer, the wings each extend from the apex. An imaginary dividing line 8 is drawn in the figures to better divide the two wings of the plate segment 3.

If the wings have projections 7, these can also support lip stimulation. In the case of recesses or notches, the flow of saliva can be diverted and/or diverted away from the central anterior space between the plate segment 3 and the lips.

A plastic material, preferably a soft material such as silicone, can be used as the preferred material for the LCT trainer shown. In this context, the following material data are preferably available, such as a hardness Shore A 40-80 (according to DIN ISO 7619-1), a tensile strength of 5 MPa-15 MPa (according to DIN 53504/ISO 37), an elongation at break of 100-1000% (according to DIN 53504/ISO 37) and/or a tear propagation resistance of 10-20 N/mm (according to ISO 34-1 method B (b) (Graves))

The LCT trainer 1 shown can be designed in one or more parts. The integral, preferably monolithic, design of the trainer has the advantage that unintentional detachment of parts and/or inhalation or ingestion is ruled out.

Alternatively, the LCT trainer 1 can also be designed in several parts, so that in particular the projections 5 are formed by interchangeable elements. For example, it is possible for the projections 5 to be arranged as receiving openings of the plate segment made of the aforementioned material with the aforementioned material data.

The interchangeable elements for the projections can be made of a different, preferably harder and/or more rigid material. This allows the lip stimulation to be adapted to the individual patient according to the developmental progress of the user's behavior, muscular development, and/or age group of the user. The adaptation can be carried out by the user himself or, in particular, by specialist personnel, such as a speech therapist or a speech therapy assistant.

In this case, the non-interchangeable unit comprising the plate segment and/or bite strip of the LCT trainer can also be available in several sizes, so that they are adapted to a child's and an adult's dentition, for example. In particular, the bite strip can also be designed in such a way that it can be extended, e.g., telescopically and/or by means of latching elements in order to adapt the bite strip to different jaw sizes.

The projections 5 can have movable secured elements, e.g., balls. Individual segments of the projections 5 also form the socket for the balls, so that they are held in a form-fit manner. The locking mechanism ensures that the movable elements cannot be accidentally loosened or swallowed. In particular, the elements are rotatably mounted relative to the immovable parts of the LCT trainer, e.g., the plate segment 3.

Particularly preferably, less than 50% of the surface of the balls or other movably secured elements can be exposed in relation to the remaining surface of the projections.

The movably mounted elements serve to further stimulate the lips and can also train movement sequences of the tongue and other body elements in the mouth area, e.g., when learning to speak.

Alternatively or additionally, the projections 5 of both the integral version and the modular version of the LCT trainer can also have recesses or notches instead of the projections 5, which are used to attach other elements. Preferably, these are elements for protecting the teeth, such as a tooth guard in sports, molded from a hard material, such as a thermosetting or thermoplastic material.

Alternatively or additionally, the projections 5 in both the integral variant and in the modular variant of the LCT trainer, in particular with interchangeable projections 5, can also have a material containing at least one active substance and/or flavor, at least one sensor element or at least one electrode.

The active ingredient-containing material can support the stimulant in particular as an active ingredient depot. In the case of a necessary but painful application, however, the active ingredient can also be an analgesic or relaxant.

It has also been shown that a positive taste sensation stimulates increased lip movement and thus an increased training effect is achieved. A typical flavoring can be, for example, a fruit acid ester, sugar or another sweetener, especially a tooth-friendly sweetener such as xylitol, which is known not to cause tooth decay.

The use of sensor elements has the special effect that, for example, when using biosensors, an inflammation status can be detected by the concentration of individual saliva ingredients. A conductivity measurement and/or other physical measurements, such as a temperature measurement, can be used to determine the oral cavity temperature, the moisture status in the interstitial space and, optionally, the extent of blood flow to the lips.

Electrodes can emit stimulation signals, such as stimulation current, to stimulate blood circulation and the muscles.

It is understood that the individual elements can also be advantageously combined with each other, so the simulant signals can stimulate the flow of saliva and/or the release times of the active ingredients.

The sensor elements can also detect when an active ingredient has been used up and needs to be replaced or whether the stimulation signals are causing inflammation or excessive unwanted heating due to incorrect settings. However, the same also applies to detecting the success of the therapy, as tissue with a higher blood supply generally has a higher temperature and/or better temperature distribution.

In the case of electrically operated sensor elements or electrodes, an energy storage unit and/or a regulating and/or evaluation unit can be arranged inside the LCT trainer, which is preferably encased in the material of the LCT trainer and therefore does not come into contact with saliva. Due to the high mechanical load on electronic components when the LCT trainer is used as intended, a multi-layer coating made of material of different hardnesses can also be provided.

If further analysis, e.g., of saliva data, is desired, the LCT trainer 1 can have collection openings that allow saliva to be sampled. If the LCT trainer 1 is coupled to a recharging station after training, this recharging station can also analyze the collected saliva samples.

The LCT trainer 1 can be adapted to different sizes for different dentitions, i.e., children's teeth, mixed dentition (from milk teeth to permanent teeth), permanent teeth and dentures. Only the length of the bite strips 2 and 3 can be adjusted distally.

The bite strips can be extended in various ways, preferably as a telescopic version, by attaching a plug-in mechanism, by screwing, locking, bayonet, cone, click and/or die system and/or particularly preferably by a plug-in cone with locking.

These different sizes of extensions can be color-coordinated with the dimensions of the teeth.

This extension can also be used to contain and/or dispense sensors (e.g. lateral flow tests), active ingredient depots, filters and/or other components or chemicals.

In addition to the electrodes, other actuators, e.g., for stimulation by vibration, preferably of the muscles, nerve stimulation and/or the glandular tissue, e.g., of the parotid gland, may alternatively or additionally be provided. In general, the sensors can receive user feedback of the reactions triggered by actuators.

As described above, sensors, active ingredient depots, actuators or user feedback attached to or incorporated in the LCT trainer can together have the effect of e.g. stimulation by vibration (actuator) and, if a biomarker (sensor system) is present, of delivering an active ingredient, in particular in doses.

Preferably, in addition to the projections 5, other elements or areas of the LCT trainer can also be used to arrange the aforementioned actuators, sensors, active ingredient depots, etc.

The extension of the bite splints 2 can also be used for active ingredient depots or other elements that are triggered by the following mechanisms, for example:
Mechanical-physical
Electric
Chemical.
Preferably: by physical action on the extension

The active ingredients can enter the body in such a way that different delivery mechanisms can be present. For chronic pain, this can be a latent release or “leaching” through osmosis or migration, or for acute treatments a release or “leaching” via porous openings through capillary forces and/or potential equalization.

The LCT trainer 1 can have permanently integrated or interchangeable elements on the lip side for tooth protection, tissue regeneration, drug delivery, sensor technology (e.g., to analyze ambient air and/or actuators (e.g. through vibration to stimulate the lips or cheeks)).

Permanently integrated or interchangeable elements for tooth coloration, enamel and gum regeneration, sensor technology and/or active substance delivery can be arranged on the tooth side.

Additional functions can also be attached to the base body, preferably by means of electrical contact elements, foils, or printing, which can record data on the state of health.

In particular, these can be mechanical, electrochemical, physical, and/or biological sensors and/or sensor elements that automatically record data at a defined interval over a defined period of time.

Since such sensors are active sensors that require power, the base body can also contain an energy source, energy cell or energy harvesting device.

Preferably, these are passive electrochemical cells, thermoelectric elements and/or induction-charged accumulators or capacitive electrical storage devices. Particularly in the case of elements that are arranged in the form of inlays on or in the LCT trainer 1, these can be replaced and inserted into the LCT trainer as required.

In addition, the sensors can be coupled directly with suitable external devices in order to address a control system, sound wave line and/or programs by means of sensor technology.

In addition, the external base station can be used to store the LCT as well as to charge the LCT and/or read out the sensors. Furthermore, the external base station can transmit the data to a central storage unit, e.g., cloud storage or/and relay station, such as smartphones. Preferably, the external base station serves as a storage box and battery station when traveling.

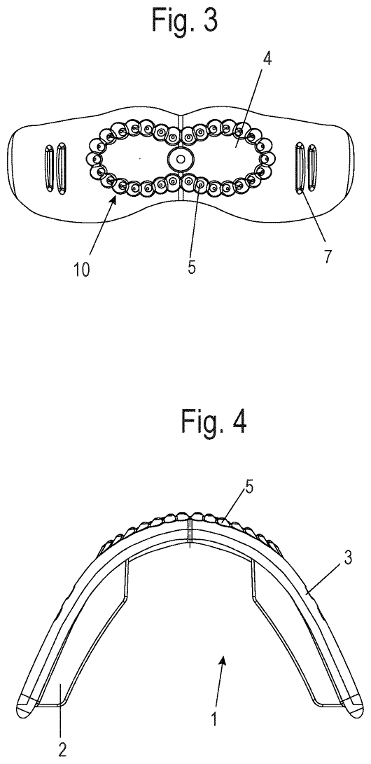

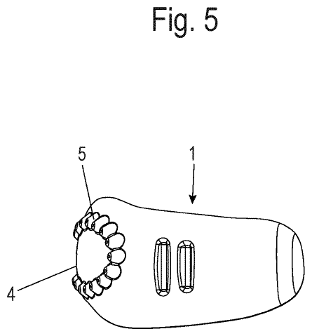

As can be seen in FIGS. 1-5, the plate segment 3 extends over both the anterior teeth of the upper and lower jaw. In contrast to a pure upper or lower jaw fixation, the variant of bilateral fixation by the upper and lower jaw teeth also allows the mouth to be opened, with the plate serving as a guide for the teeth. This allows the LCT trainer according to the invention to be held securely in the oral cavity during speech or other activities.

The bite strip 2 lies on a bite-on plane, which preferably runs through the center of the plate segment. The projections 5 lie both above and below the bite-on plane on the plate segment.

This not only trains individual muscle parts of the upper or lower jaw muscle, but also the entire orbicularis oris muscle, i.e., the mouth ring muscle.

Holistic training of the upper and lower lip muscles as well as the cheek muscles can activate drooping upper or lower lips and also train the entire lip closure.

The LCT trainer optionally has a non-displayed light source, e.g., a UV light source, preferably in the form of an LED light source. This can be used for tooth bleaching, for example. A corresponding power supply source, such as a battery or a power connection, can be provided.

Other sensors or actuators can be integrated in the LCT trainer. Furthermore, a transmitter and/or receiver module is preferably integrated in the LCT trainer, which can establish a connection with an external device, e.g., a smartphone, to control the actuators, e.g. the light source and/or to evaluate the sensor data.

Although the invention has been illustrated and described in detail by way of preferred embodiments, the invention is not limited by the examples disclosed, and other variations can be derived from these by the person skilled in the art without leaving the scope of the invention. It is therefore clear that there is a plurality of possible variations. It is also clear that embodiments stated by way of example are only really examples that are not to be seen as limiting the scope, application possibilities or configuration of the invention in any way. In fact, the preceding description and the description of the figures enable the person skilled in the art to implement the exemplary embodiments in concrete manner, wherein, with the knowledge of the disclosed inventive concept, the person skilled in the art is able to undertake various changes, for example, with regard to the functioning or arrangement of individual elements stated in an exemplary embodiment without leaving the scope of the invention, which is defined by the claims and their legal equivalents, such as further explanations in the description.

### REFERENCE SIGNS

1 LCT trainer
2 Bite strip
3 Plate segment
4 Retaining surface
5 Projection
6 Central surface
7 Projection
8 Dividing line
9 Central surface
10 Arrangement of several projections

1-12. (canceled)

13. A logopaedic treatment device comprising:
a U-shaped basic shape configured for insertion in a mouth;
a plate segment configured to partially cover rows of teeth of an upper and lower jaw in the mouth; and
at least one bite strip configured for the teeth of the upper and lower jaw to bite on,
wherein the plate segment comprises, on a side facing away from the teeth, an arrangement of a plurality of projections configured to stimulate a lip of the mouth,
wherein the bite strip defines a bite-on plane, and
wherein projections of the plurality of projections are arranged both above and below the bite-on plane.

14. The logopaedic treatment device of claim 13, wherein the plurality of projections of the plate segment comprises at least 5 projections configured to stimulate the lip.

15. The logopaedic treatment device of claim 13, wherein the plurality of projections have a height of greater than 0.5 mm and have a cylindrical or frustoconical shape.

16. The logopaedic treatment device of claim 13, wherein the plurality of projections comprise movable elements.

17. The logopaedic treatment device of claim 13, wherein the plurality of projections comprise sensor elements or actuators.

18. The logopaedic treatment device of claim 13, further comprising:
a retaining surface configured for clamping or latching retention of an active substance depot or flavoring substance depot.

19. The logopaedic treatment device of claim 13, wherein the plurality of projections comprise an active substance, a flavoring substance, or an indicator substance.

20. The logopaedic treatment device of claim 13, further comprising:
a telescopic extension configured to adapt the logopaedic treatment device to a size of the teeth of the mouth.

21. The logopaedic treatment device of claim 13, wherein the logopaedic treatment device is a monolithic single piece.

22. The logopaedic treatment device of claim 13, wherein the logopaedic treatment device has a modular structure, wherein the plurality of projections are interchangeably arranged on the plate segment.

23. The logopaedic treatment device of claim 13, wherein the logopaedic treatment device is formed, at least in some areas, from a material with a Shore hardness A of 40-80.

24. The logopaedic treatment device of claim 13, wherein the plate segment, beyond the plurality of projections and on a side facing the teeth, has regions with different surface roughness.
